(19) United States
(12) Patent Application Publication
(10) Pub. No.: US 2005/0119593 A1
(43) Pub. Date: Jun. 2, 2005

(54) Method of viewing and controlling balance of the vertebral column

(75) Inventors: Emeric Gallard, Paris (FR); Guy Viart, Saint-Leger (FR)

Correspondence Address: YOUNG & THOMPSON, 745 SOUTH 23RD STREET, 2ND FLOOR, ARLINGTON, VA, 22202, US

(73) Assignee: EUROSURGICAL SA, BEAURAINS (FR)

(21) Appl. No.: 10/506,564

(22) PCT Filed: Mar. 4, 2003
(86) PCT No.: PCT/FR03/00694

(30) Foreign Application Priority Data
Mar. 5, 2002 (FR) 02/02754

(51) Int. Cl. A61B 5/107

(52) U.S. Cl. 600/594

(57) ABSTRACT

The invention relates to a method of viewing and controlling the balance of a vertebral column of which one spinal segment is corrected by way of conventional spinal instrumentation. The invention method consists of: determining and calculating the relative three-dimensional position of the upper (UEV) and lower (LEV) end instrumented vertebrae of the corrected spinal segment; determining and calculating the position of the spinal segments which are located above and below the spinal segment corrected by the spinal instrumentation, according to the relative three-dimensional position of the upper (UEV) and lower (LEV) end instrumented vertebrae and viewing the front and side projection of the balance or imbalance of the vertebral column.

The present invention relates to a method of viewing and controlling the balance of a vertebral column of a patient.

The viewing and controlling method according to the present invention is more particularly designed for viewing and controlling the balance of the vertebral column of a patient during an operation on a spinal segment corrected by means of conventional spinal instrumentation.

The osteosynthesis instrumentation or equipment known to date are generally comprised of bone anchoring screws, connectors fixed by the screws on the vertebral body of each vertebra and connection rods interconnecting the connectors. Sometimes, the connection rods are deformed by the surgeon so as to be able to reestablish the curvature of the spinal segment to be corrected.

The idea of balance of the vertebral column is rather complex and is characterized by different parameters depending on the clinical approach.

Most of the time surgeons evaluate spinal balance both clinically and by radiological photography.

In the latter case, two large-size (30 cm×90 cm) radiographical images are taken, one allowing the side of the patient's vertebral column to be viewed and the other the front.

Overall, it is considered that the frontal balance of the vertebral column of the patient is obtained from the moment where the first thoracic vertebra (T1) is centered with comparison to the sacrum.

As regards sagittal balance, it is considered to be obtained from the moment where the external auditory canals are situated vertically from the femoral heads.

When surgical intervention in the vertebral column is necessary, the simple act of interfusing the vertebrae through instrumentation may cause post-operative balance problems to the patient.

The objective of the surgeon is clearly to first reduce the curvature of the spine in the case of spinal deformation and/or to attach a zone that will be instrumented.

Subsequently, the surgeon must make sure that the instrumented zone allows the patient to gain or regain his balance after the operation, when he will take a standing position.

Thus, during the operation, the surgeons attempt to evaluate a possible post-operative balance or imbalance in the patient.

Currently surgeons have no system at their disposal that allows them to know if the instrumented level is going to permit the post-operative balance of the patient.

Surgeons thus use a single visual assessment of the instrumented segment in the operative field. Some use a radiographic means (two Rx front+side images) that allows them to see a larger view of the vertebral column (from the femoral heads to the cervical vertebrae).

The surgeons may then assess not only the instrumented segment but also the segments overlying and underlying the instrumentation in the frontal and sagittal planes.

Unfortunately, these radiographs are performed on the patient who is lying down on the operating table, and consequently may not systematically predict the behavior of the operated vertebral column when the patient stands up after surgery.

The problem consists in that the patient is recumbent on the operating table, and only the vertebrae that will be fused are cleared by a conventional posterior surgical approach.

The surgeon instruments these vertebrae, and fuses the same, but may not be able to assess the consequence of this surgical act on the underlying and overlying segments that clearly are adapted to the new geometry that the surgeon gives to the segment that he instruments.

It is noted that in each instrumentation there exists an upper end instrumented vertebrae (UEV) and a lower end instrumented vertebrae (LEV) that are respectively connected naturally to the vertebrae of the non-instrumented vertebral column, that are located above and below the instrumentation.

It is the orientation of the upper end-plate of the first instrumented vertebra (UEV) compared to the lower end-plate of the last instrumented vertebra (LEV) that will define the adaptation behavior of the underlying and overlying segment.

Consequently, it is the relative position of the two instrumented vertebrae ends that will determine the geometry of the overlying and underlying segments.

The instrumented segment is transformed into a large vertebra whose “edges” will determine the adaptation behavior of the overlying and underlying segments and thus determine the postoperative balance or imbalance of the patient in a standing position.

The object of the method according to the present invention is to determine the balance of the vertebral column by focusing on the two vertebrae extremes (UEV and LEV).

The method according to the present invention allows the virtual column of the patient in a standing position after an operation to be seen on a control screen.

For that, measurements must be -taken on the end instrumented vertebrae (UEV and LEV).

Measurements taken only on these vertebrae will allow the measuring time, which is always too long in these often tedious operations, to be limited.

From the measurements taken on these end instrumented vertebrae, calculation algorithms will allow the balance possibilities of the standing patient after surgery to be projected on the preoperative radiographs of the patient.

Thus, the present invention provides a method of viewing and controlling the balance of the vertebral column wherein one spinal segment is corrected by means of conventional spinal instrumentation, characterized in that it consists of:
determining and calculating the relative three-dimensional position of the upper (UEV) and lower (LEV) end instrumented vertebrae of the corrected spinal segment through anatomical points or contours identified or digitalized on the radiographs of the patient to be treated,
determining and calculating the position of the spinal segments located above and below the spinal segment corrected by the spinal instrumentation, according to the relative three-dimensional position of the upper (UEV) and lower (LEV) end instrumented vertebrae, through anatomical points or contours identified or digitalized on the radiographs of the patient to be treated,
and viewing the balance or imbalance of the vertebral column (1) in the vertical position and in front and side projection.

According to an embodiment of the present invention, the relative three-dimensional position of the upper (UEV) and lower (LEV) end instrumented vertebrae of the corrected spinal segment is obtained from a first reconstruction in a three-dimensional visual space of the geometry of the external envelope or contour of the upper (UEV) and lower (LEV) end instrumented vertebrae and a second reconstruction in a three-dimensional visual space of the surface of the neural arch of the (UEV) and lower (LEV) end instrumented vertebrae.

According to an embodiment of the present invention, the first and second reconstruction steps in a three-dimensional visual space allow the relative position of the upper (UEV) and lower (LEV) end instrumented vertebrae of the corrected spinal segment to be determined in a three-dimensional visual space.

According to an embodiment of the present invention, the method of viewing and controlling consists in that the upper (UEV) and lower (LEV) end instrumented vertebrae of the corrected spinal segment are projected onto front and side radiographs of the patient to be treated.

According to an embodiment of the present invention, the method of viewing and controlling consists in that the front and side projection of the upper (UEV) and lower (LEV) end instrumented vertebrae of the corrected spinal segment allows the position on the front and side radiographs of the spinal segments located above and below the corrected spinal segment to be determined, and to view the appearance of the reconstructed vertebral column on the front and side radiographs.

According to an embodiment of the present invention, the method of viewing and controlling consists of identifying or digitalizing at least four points delimiting a rectangle reproducing the vertebral body for each vertebra of the vertebral column.

According to an embodiment of the present invention, the method of viewing and controlling consists of identifying or digitalizing the points that correspond to the radiological indicators utilized, to define the balance of the head of the patient compared to the pelvis.

According to an embodiment of the present invention, the method of viewing and controlling consists of identifying or digitalizing at least ten points of the head allowing the external contour of the head to be marked.

According to an embodiment of the present invention, the method of viewing and controlling consists of identifying or digitalizing for the pelvis at least the anatomical points defining the center of each femoral head and the sacral plate.

According to an embodiment of the present invention, the method of viewing and controlling consists of identifying or digitalizing at least five points of the pelvis wherein one is for each femoral head and at least three are for the sacrum, in order to form a triangle.

According to an embodiment of the present invention, the method of viewing and controlling consists of embedding the digitalized points in the radiographs of the patient.

According to an embodiment of the present invention, the method of viewing and controlling consists of reconstructing in three dimensions the geometric form of the upper (UEV) and lower (LEV) end instrumented vertebrae from the sagittal and frontal radiographs of the patient.

According to an embodiment of the present invention, the method of viewing and controlling consists of determining the linear and angular geometric position of the lower (LEV) end instrumented vertebrae, reconstructed with relation to the front and side radiographs.

According to an embodiment of the present invention, the method of viewing and controlling consists of projecting the upper (UEV) and lower (LEV) end instrumented vertebrae onto the front and side radiographs.

According to an embodiment of the present invention, the method of viewing and controlling consists of embedding the projection of the upper (UEV) and lower (LEV) end instrumented vertebrae, with relation to one another, in the radiographs, by registration of the projection of the lower end instrumented vertebra (LEV).

The characteristics and advantages of the present invention will be shown in detail in the following description of the particular embodiments in a non-limiting manner with relation to the appended figures.

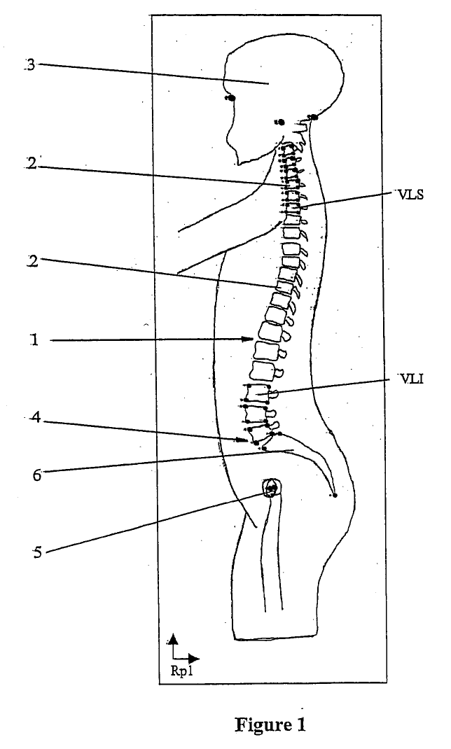

FIG. 1 represents a sagittal radiograph of a standing patient, on which the anatomical points are digitalized to identify the pelvis and the femoral heads.

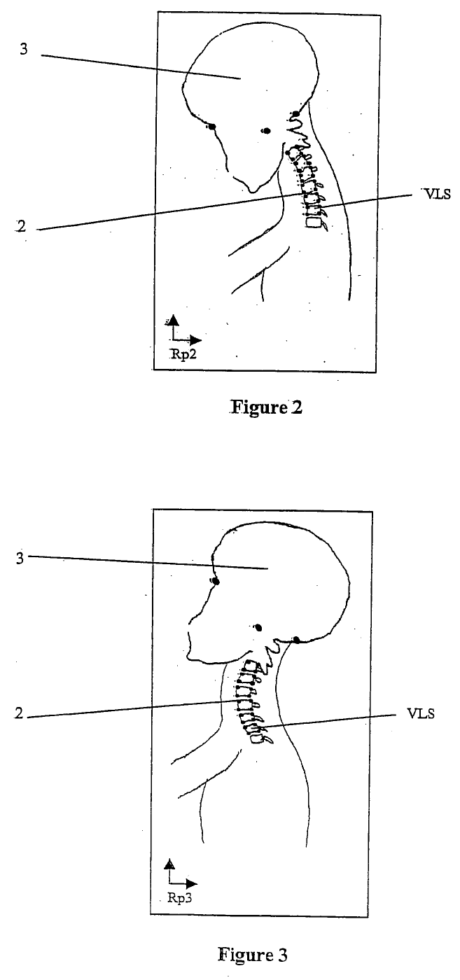

FIG. 2 illustrates a sagittal radiograph of a standing patient in-cervical flexion, on which the anatomical points are digitalized to identify the head, the vertebrae overlying the instrumentation and the upper end instrumented vertebrae (UEV).

FIG. 3 shows a sagittal radiograph of a standing patient in cervical flexion, on which the anatomical points are digitalized to identify the head, the vertebrae overlying the instrumentation and the upper end instrumented vertebrae (UEV).

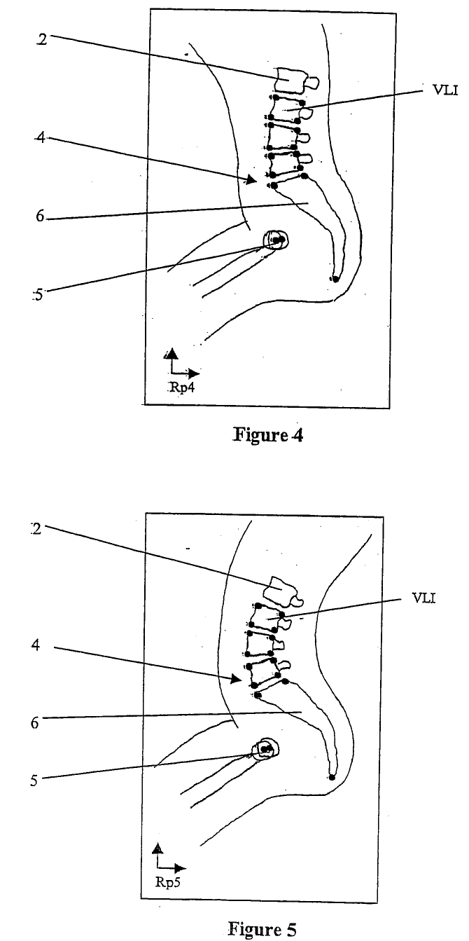

FIG. 4 represents a sagittal radiograph of a seated patient in lumbar flexion, on which the anatomical points are digitalized to identify the pelvis, the vertebrae underlying the instrumentation and the lower end instrumented vertebrae (LEV).

FIG. 5 represents a sagittal radiograph of a seated patient in lumbar extension, on which the anatomical points are digitalized to identify the pelvis, the vertebrae underlying the instrumentation and the lower end instrumented vertebrae (LEV).

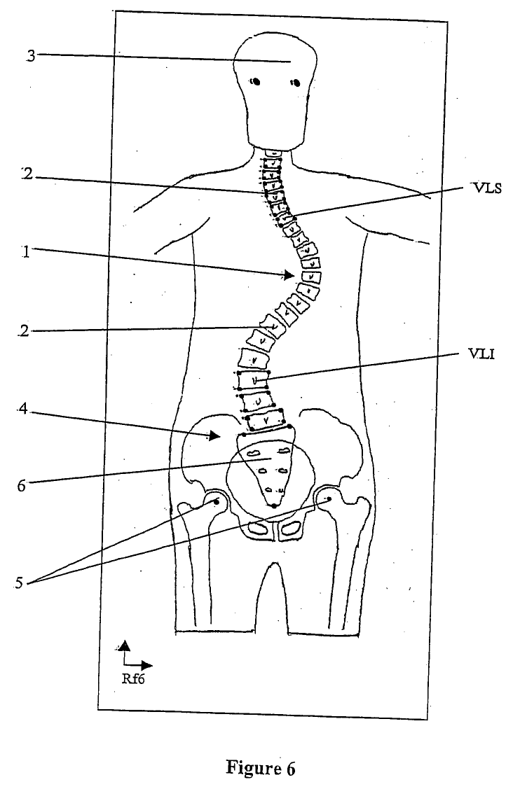

FIG. 6 illustrates a frontal radiograph of a standing patient, on which the anatomical points are digitalized to identify the pelvis and the femoral heads.

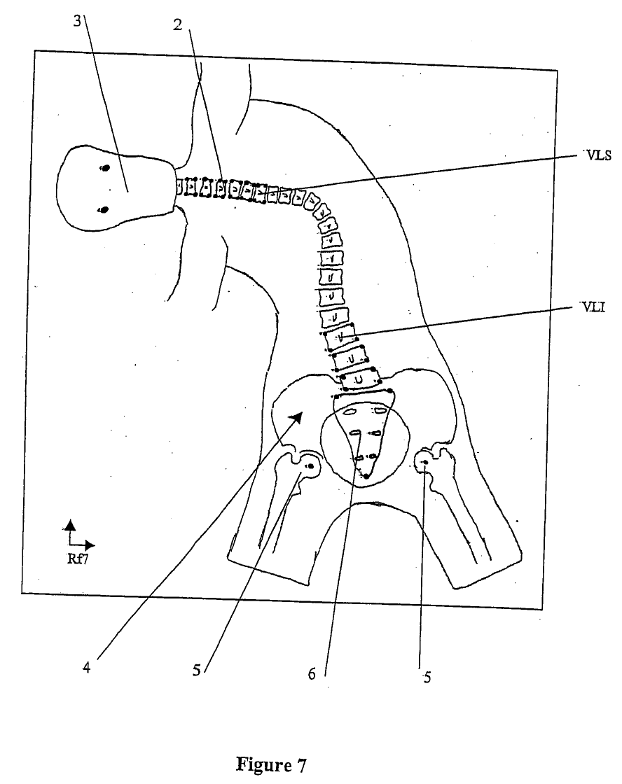

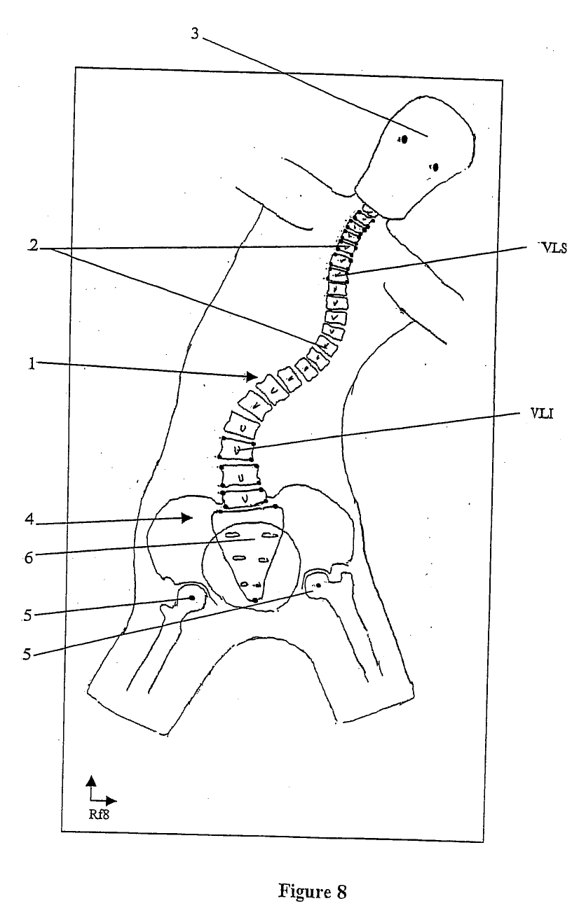

FIG. 7 shows a frontal radiograph of a patient who is lying down in lateral inflexion to the left, on which the anatomical points are digitalized to identify the head, the vertebrae overlying and underlying the instrumentation, the upper (UEV) and lower (LEV) end instrumented vertebrae, the pelvis and the femoral heads.

FIG. 8 represents a frontal radiograph of a patient who is lying down in lateral inflexion to the right, on which the anatomical points are digitalized to identify the head, the vertebrae underlying and overlying the instrumentation, the upper (UEV) and lower (LEV) end instrumented vertebrae, the pelvis and the femoral heads.

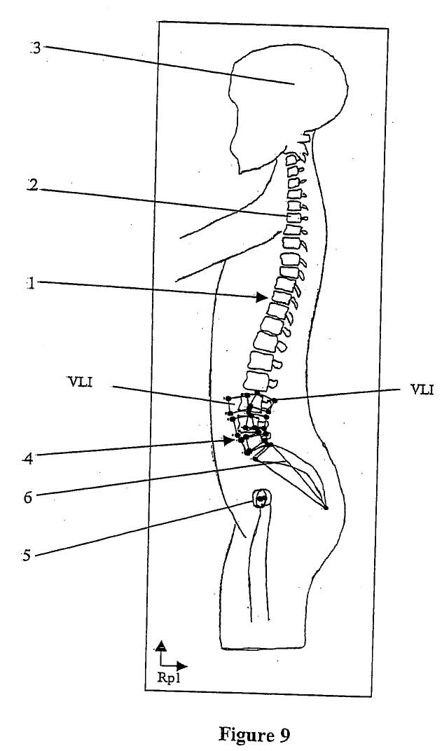

FIG. 9 illustrates the radiograph of FIG. 1 in which the digitalized anatomical points have been embedded in the radiographs of FIGS. 4 and 5, this after mapping the scales and adjustment with relation to the pelvis.

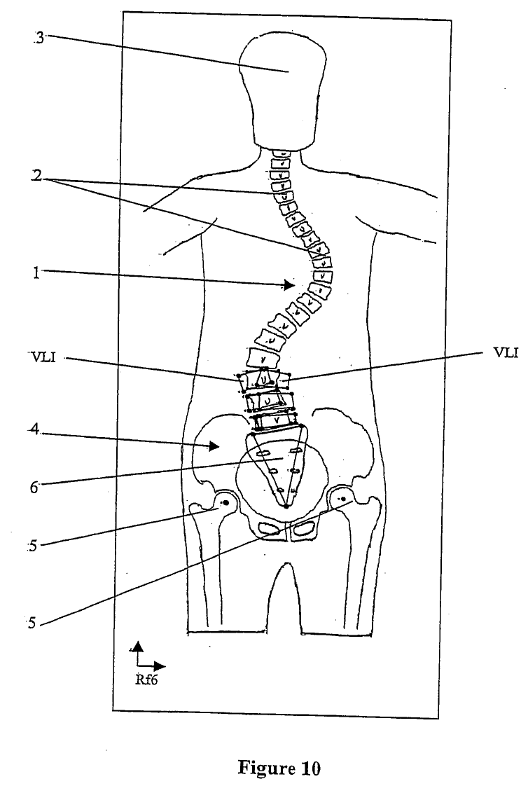

FIG. 10 shows the radiograph of FIG. 6 in which the digitalized anatomical points have been embedded in the radiographs of FIGS. 7 and 8, (only those representing the pelvis, the vertebrae underlying the instrumentation, and the lower (LEV) end instrumented vertebrae), after mapping the scales and adjustment with relation to the pelvis.

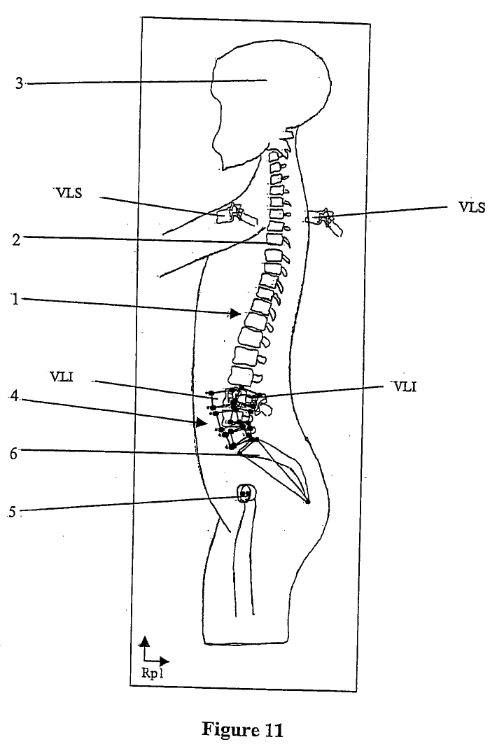

FIG. 11 illustrates the radiograph of FIG. 9 in which the projection of the upper (UEV) and lower (LEV) end instrumented vertebrae has been embedded by registration of the lower end instrumented vertebrae (LEV).

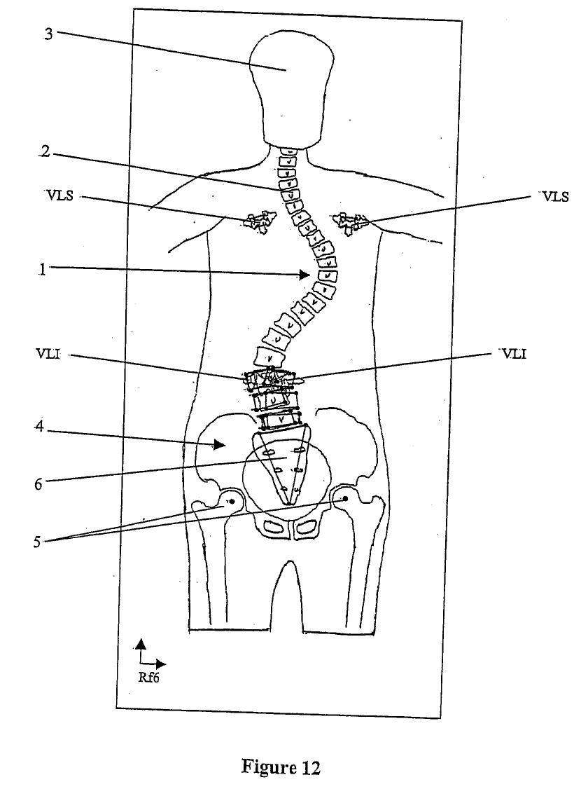

FIG. 12 shows the radiograph of FIG. 10 in which the projection of the upper (UEV) and lower (LEV) end instrumented vertebrae has been embedded by registration of the lower end instrumented vertebrae (LEV).

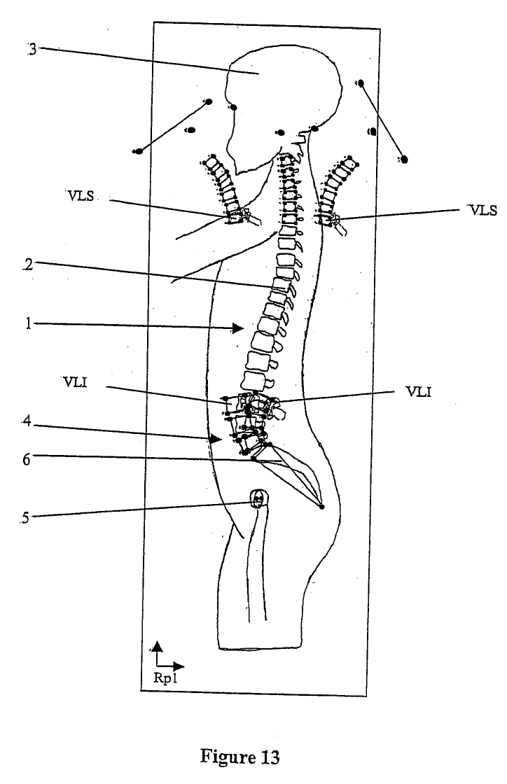

FIG. 13 represents the radiograph of FIG. 11 in which the digitalized points have been embedded in the radiograph of FIGS. 2 and 3 (head, overlying vertebrae and upper end instrumented vertebrae (UEV)), by registration of the upper end instrumented vertebrae (UEV).

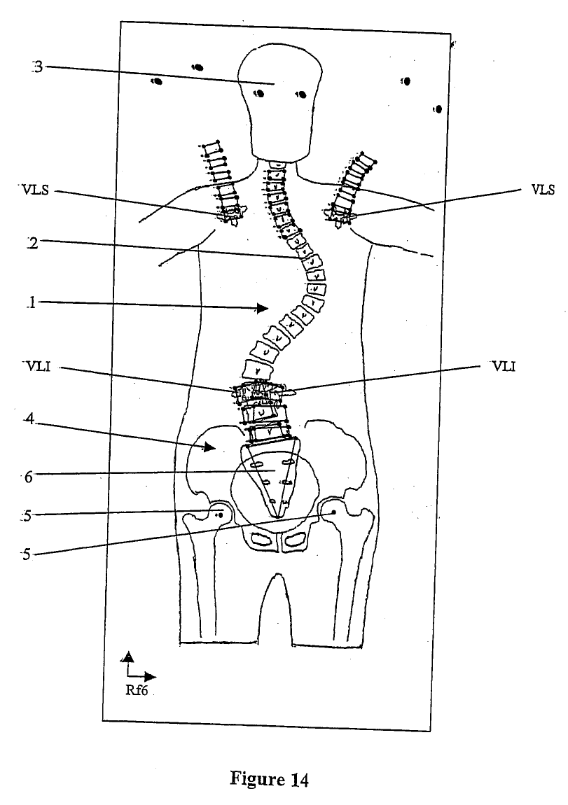

FIG. 14 illustrates the radiograph of FIG. 12 in which the digitalized points have been embedded in the radiograph of FIGS. 7 and 8 (head, overlying vertebrae and upper end instrumented vertebrae (UEV)), by registration of the upper end instrumented vertebrae (UEV)).

### First Step of the Method

The first step of the method is to identify or digitalize the anatomical points or contours on the radiographs of the patient to be treated.

Thus for each vertebra 2 of the vertebral column 1, it is necessary to identify at least four points. These latter delimit a rectangle reproducing the vertebral body.

For the head 3, it is necessary to identify the anatomical points that usually correspond to the radiological indicators utilized for defining the balance of the head 3 of the patient with relation to the pelvis 4.

For the head 3, it is necessary to identify in a non-limiting manner, at least ten points that allow the external contour of the head to be marked.

For the pelvis 4, it is necessary to identify at least the anatomical points defining the center of each femoral head 5 and the sacrum 6.

For the pelvis 4, it is necessary to identify at least five points wherein one is for each femoral head 5 and at least three are for the sacrum 6 in order to form a triangle.

It is noted that the two-dimensional coordinates (x, y) of each point are known in the indicator connected to the digitalized radiograph.

This first step is illustrated on each of FIGS. 1 to 8.

### Second Step of the Method

The second step consists of embedding the digitalized points in the radiographs of FIGS. 4 and 5 in the radiograph of FIG. 1.

During this embedding of the digitalized points or contours, it is necessary to adjust the scale between the radiographs and to superimpose the points defining the pelvis 4 by registration (FIG. 9).

Also, embedding of the points digitalized on the radiographs of FIGS. 7 and 8 is carried out in the radiograph of FIG. 6.

The digitalized points or contours relate more particularly to those representing the pelvis 4, the vertebrae underlying the instrumentation, and the lower end instrumented vertebrae (LEV). A mapping of the scales and an adjustment or registration of the points or contour with relation to the pelvis 4 (FIG. 10) is provided in this step of the method.

### Third Stop of the Process

This step consists of a three-dimensional reconstruction of the geometric form of the vertebrae 2 and more particularly the end instrumented vertebrae from the sagittal (FIG. 1) and frontal (FIG. 6) radiographs, that is:
the upper end instrumented vertebrae (UEV),
and the lower end instrumented vertebrae (LEV).

This step also consists of determining the linear and angular geometric position of the lower end instrumented vertebrae (LEV) reconstructed with relation to the front and side radiographs (positioning of the patient with relation to the films (Rx) when radiographic images are taken).

During this step it is necessary to:represent the geometric indicators, that is:
a three-dimensional indicator is associated with the three-dimensional geometry of the upper end instrumented vertebrae (UEV),
a three-dimensional indicator is associated with the three-dimensional geometry of the upper [sic] end instrumented vertebrae (LEV),
a two-dimensional indicator is associated with the projection of -the geometric form of the upper end instrumented vertebrae (LEV) [sic] on the side radiograph (FIG. 1),.
a two-dimensional indicator is associated with the projection of the geometric form of the upper end instrumented vertebrae (LEV) [sic] on the front radiograph (FIG. 6).

### Fourth Step of the Method

This step consists of reconstructing the geometry of the external envelope or contour of the upper (UEV) and lower (LEV) end instrumented vertebrae in a three-dimensional visual space.

During this step, a second reconstruction of the surface of the neural arch of the upper (UEV) and lower (LEV) end vertebrae is provided in a three-dimensional visual space.

### Fifth Step of the Method

This step consists of projecting the upper (UEV) and lower (LEV) end instrumented vertebrae on the front and side radiographs thanks to the relationship established between the three-dimensional and two-dimensional indicators defined during the third step.

This step also consists of embedding the projection of the upper (UEV) and lower (LEV) end instrumented vertebrae with relation to one another in the radiographs of FIGS. 9 and 10 by registration of the projection of the lower end instrumented vertebrae (LEV) on the two positions this vertebra occupies in the radiographs of FIGS. 9 and 10.

FIGS. 11 and 12 illustrate this step.

### Sixth Step of the Method

This step consists of embedding the digitalized points or contours in the radiograph of FIG. 11 in the radiographs of FIGS. 2 and 3 by adjusting the scale between the radiographs and registration with relation to the upper end instrumented vertebrae (UEV).

In the same manner, the points or contours digitalized on the radiographs of FIGS. 7 and 8 are embedded in the radiograph of FIG. 12, by adjusting the scale between the radiographs and registration with relation to the upper end instrumented vertebrae (UEV).

FIGS. 13 and 14 illustrate this step.

### Seventh Step of the Method

Based on the results obtained during the previous steps, this step consists of:
determining and calculating the relative three-dimensional position of the upper (UEV) and lower (LEV) end instrumented vertebrae of the corrected spinal segment,
determining and calculating the position of the spinal segments located above and below the spinal segment corrected by the spinal instrumentation according to the relative three-dimensional position of the upper (UEV) and lower (LEV) end instrumented vertebrae,
and viewing the balance or imbalance of the vertebral column 1 in front and side projection.

It is noted that for achieving the fourth step, the latter may be, for example, achieved from the three-dimensional emitter/sensor with a tracer pen allowing the reconstruction of the external contour and surface of the neural arch of the upper (UEV) and lower (LEV) end instrumented vertebrae in a three-dimensional visual space.

Furthermore, the implementation of the method of the invention calls for digital image processing techniques wherein the practical implementation is within reach of a person skilled in the art.

This implementation is achieved from an image processing system to allow the viewing of the results and the relative position in front and side projection of the upper (UEV) and lower (LEV) end instrumented vertebrae of the spinal segment corrected by the spinal instrumentation.

1. A method of viewing and controlling the balance of a vertebral column of which one spinal segment is corrected by means of conventional spinal instrumentation, characterized in that the method consists of:
determining and calculating the relative three-dimensional position of the upper (UEV) and lower (.LEV) end instrumented vertebrae of the spinal segment corrected through anatomical points or contours identified or digitalized on the radiographs of the patient to be treated,
determining and calculating the position of the spinal segments which are located above and below the spinal segment corrected by the spinal instrumentation, through anatomical points or contours identified or digitalized on the radiographs of the patient to be treated, according to the relative three-dimensional position of the upper (UEV) and lower (LEV) end instrumented vertebrae,
and viewing the balance or imbalance of the vertebral column (1) in the vertical position and in front and side projection.

2. The method of viewing and controlling according to claim 1, characterized in that the relative three-dimensional position of the upper (UEV) and lower (LEV) end instrumented vertebrae of the corrected spinal segment is obtained from a first reconstruction in a three-dimensional visual space of the geometry of the external envelop or contour of the upper (UEV) and lower (LEV) end instrumented vertebrae and a second reconstruction in a three-dimensional visual space of the surface of the neural arch of the upper (UEV) and lower (LEV) end instrumented vertebrae.

3. The method of viewing and controlling according to claim 2, characterized in that the first and second reconstruction steps in a three-dimensional visual space allow the relative position of the upper (UEV) and lower (LEV) end instrumented vertebrae of the corrected spinal segment to be determined in a three-dimensional visual space.

4. The method of viewing and controlling according to claim 3, characterized in that the upper (UEV) and lower (LEV) end instrumented vertebrae of the corrected spinal segment are projected on the front and side radiographs of the patient to be treated.

5. The method of viewing and controlling according to claim 3, characterized in that the front and side projection of the upper (UEV) and lower (LEV) end instrumented vertebrae of the corrected spinal segment allows the position on the front and side radiographs of the spinal segments located above and below the corrected spinal segment to be determined, and to view the appearance of the reconstructed vertebral column on the front and side radiographs.

6. The method of viewing and controlling according to claim 1, characterized in that the method consists of identifying or digitalizing at least four points delimiting a rectangle reproducing the vertebral body for each vertebra of the vertebral column.

7. The method of viewing and controlling according to claim 1, characterized in that the method consists of identifying or digitalizing the points that correspond to the radiological indicators utilized to define the balance of the head of the patient with relation to the pelvis.

8. The method of viewing and controlling according to claim 7, characterized in that the method consists of identifying or digitalizing at least ten points for the head allowing the external contour of the head to be marked.

9. The method of viewing and controlling according to claim 1, characterized in that the method consists of identifying or digitalizing at least the anatomical points for the pelvis defining the center of each femoral head and the sacral plate.

10. The method of viewing and controlling according to claim 9, characterized in that the method consists of identifying or digitalizing at least five points for the pelvis of which one is for each femoral head and at least three are for the sacrum in order to form a triangle.

11. The method of viewing and controlling according to claim 1, characterized in that the method consists of embedding the digitalized points in the radiographs of the patient.

12. The method of viewing and controlling according to claim 1, characterized in that the method consists of reconstructing the geometric form of the upper (UEV) and lower (LEV) end instrumented vertebrae in three dimensions from the sagittal and frontal radiographs of the patient.

13. The method of viewing and controlling according to claim 1, characterized in that the method consists of determining the linear and angular geometric position of the reconstructed lower end instrumented vertebrae (LEV) with relation to the front and side radiographs.

14. The method of viewing and controlling according to claim 1, characterized in that the method consists of projecting the upper (UEV) and lower (LEV) end instrumented vertebrae on the front and side radiographs.

15. The method of viewing and controlling according to claim 1, characterized in that the method consists of embedding the projection of the upper (UEV) and lower (LEV) end instrumented vertebrae in the radiographs, with relation to one another, by registration of the projection of the lower end instrumented vertebrae (LEV).
